(19) United States
(12) Patent Application Publication
(10) Pub. No.: US 2026/0085369 A1
(43) Pub. Date: Mar. 26, 2026

(54) KIT FOR RAPID DETECTION OF AVIAN LEUKOSIS VIRUS SUBGROUP J BASED ON CRISPR/Cas13a SYSTEM

(71) Applicants: Southwest University, Chongqing (CN); ChongQing Academy of Animal Sciences, Chongqing (CN)

(72) Inventors: Xi LAN, Chongqing (CN); Qigui WANG, Chongqing (CN); Haiwei WANG, Chongqing (CN); Shutao CHEN, Chongqing (CN); Min TAN, Chongqing (CN); Zhifu CUI, Chongqing (CN); Yongju ZHAO, Chongqing (CN)

(21) Appl. No.: 19/080,176

(22) Filed: Mar. 14, 2025

(30) Foreign Application Priority Data
Sep. 26, 2024 (CN) 202411350812.9

(51) Int. Cl. C12Q 1/70; C12N 9/22; C12N 15/11

(52) U.S. Cl. CPC C12Q 1/702; C12N 9/22; C12N 15/11; C12N 2310/20; C12N 2320/10; C12Q 2600/158

(57) ABSTRACT

Provided is a kit for rapid detection of avian leukosis virus subgroup J (ALV-J) based on a CRISPR/Cas13a system. The method is based on the combination of the CRISPR/Cas13a system and recombinase aided amplification (RAA) for ALV-J detection. An oligonucleotide probe is designed as a substrate for CRISPR/Cas13a trans-cleavage and produces a detectable signal. The method can substantially improve detection sensitivity by amplifying a detection signal twice by RAA and T7 transcription. The detection method further exhibits excellent specificity, allowing for clear differentiate from other avian viruses. It does not require expensive experimental equipment and special laboratory environment, and it is rapid and efficient. The method is of great significance for biological research and on-site detection of ALV-J.

### CROSS REFERENCE TO RELATED APPLICATION

This patent application claims the benefit and priority of Chinese Patent Application No. 202411350812.9 filed with the China National Intellectual Property Administration on Sep. 26, 2024, the disclosure of which is incorporated by reference herein in its entirety as part of the present application.

### REFERENCE TO SEQUENCE LISTING

A computer readable XML file entitled “GWP20241208098_seqlist”, that was created on Jan. 7, 2025 with a file size of about 28,030 bytes, contains the sequence listing for this application, has been filed with this application, and is hereby incorporated by reference in its entirety.

### TECHNICAL FIELD

The present disclosure relates to the field of detection, in particular to a kit for rapid detection of avian leukemia virus subtype J (ALV-J) based on a CRISPR/Cas13a system.

### BACKGROUND

Avian leukemia virus (ALV) belongs to the genus Alpharetrovirus of the family Retroviridae. It is characterized by sporadic onset, with a morbidity rate of 3-25%. ALV was first discovered by Rous in the UK in 1911. Since then, its presence has been detected in chicken farms worldwide. ALV infection can cause immunosuppressive diseases in chickens, inhibit chicken's growth, and induce tumors that may lead to death, with a mortality rate of 1-2%, and even 20% in some chicken farms with poor environment. Despite more than a century of research, there is still no vaccine to protect chickens from ALV infection.

ALV is divided into 11 subgroups (subgroups A to K) according to the host range, the interaction between different virulent strains, and the antigenic structure of virus envelope glycoprotein, among which subgroup E belongs to endogenous ALV, and is either non-pathogenic or has low pathogenicity. In 1991, Payne discovered and isolated a new subgroup of ALV and named it subgroup J. ALV Subgroup J (ALV-J) can induce various types of tumors in chickens, mainly bone marrow cell-like tumors. Unlike other ALV subgroups, ALV-J exhibits much higher infectivity and pathogenicity than canonical ALV subgroups A, B, C, and D. In the mid-1990s, ALV-J had spread to almost all countries and infected nearly all white feather broiler flocks globally, leading to substantial losses to the global poultry husbandry. The transmission of ALV-J occurs through both horizontal and vertical routes. Once a chicken farm is infected with ALV-J and fails to implement timely purification, all parental and commercial chicken flocks are likely to be infected. This situation can lead to the rapid spread of the viruses throughout the chicken flock, causing serious economic losses and public health risks to the breeding industry. Therefore, timely detection and purification of ALV-J is crucial to ensure the health of chicken flocks and maintain production efficiency.

Given that the disease caused by ALV-J infection usually breaks out in local breeding chicken farms, and is propagated by super-large chicken farms, carly diagnosis of ALV-J is particularly important in prophylaxis and control efforts. Existing methods for determining ALV-J infection include polymerase chain reaction (PCR), enzyme-linked immunosorbent assay (ELISA), indirect immunofluorescence assay (IFA), virus isolation and detection, immunohistochemical techniques for detecting ALV-J in tissue samples, as well as nucleic acid techniques for detecting pathogenic exogenous ALV-J, Techniques using reverse transcription polymerase chain reaction (RT-PCR) and nucleic acid probe hybridization are also employed to detect ALV-J. Among them, the specific antibody immunohistochemistry method for detecting ALV-J in specimens is cumbersome and technically challenging, and only a limited number of specimens can be selected. The virus isolation method is recognized as the international gold standard for virus detection. This method is mainly achieved by inoculating the collected plasma into cells, culturing them for 7-9 days, and using ALV-J ELISA antigen kit for detection. However, this method has a long detection cycle and a high cost, and requires sophisticated instruments and skilled operators, which makes it difficult to deploy in places far away from farms. Therefore, it is very important to develop a virus detection method with high sensitivity and specificity, fast detection speed, and casier operation to promote the detection of ALV-J in remote areas.

CRISPR (clustered regularly interspaced short palindromic repeats) is a self-protective mechanism found in archaebacteria, known as an adaptive immune system, which was first discovered in 1987. The CRISPR systems are divided into two classes based on different components and mechanisms of action: Class 1 systems composed of polyprotein complex effectors, and Class 2 systems composed of single effector proteins. Class 1 system includes types I, III and IV, and Class 2 system includes types II, V, and VI. So far, at least 26 subtypes have been discovered, and different types and subtypes are rapidly expanding. Two Class 2 RNA-guided endonucleases, Cas12a and Cas13a, have been used as biosensors to target single-stranded or double-stranded DNA and single-stranded RNA, respectively. Cas13 forms a complex with crRNA to recognize targeted ssRNA, thereby activating the HPEN domain in the Cas 13a protein, which can cleave any ssRNA in the environment.

Due to the specific cleavage function and convenience of detection, the CRISPR-Cas13a system has been applied to detect various diseases and some biomarkers. Zheng Feng et al. developed a SHERLOCK system for target nucleic acid detection using such a CRISPR-Cas13 signal transduction process, in order to specifically detect Zika virus. CrRNA determines the specificity of CRISPR detection. Parinaz Fozouni et al. designed crRNAs that bind to a plurality of regions of SARS-COV-13 RNA to improve the sensitivity of CRISPR detection. In addition, the detection of Nipah virus (NiV) based on CRISPR/Cas13a system and the detection of fowl adenovirus in serum were also realized by using lateral flow assay. In order to solve the background interference during RNA amplification, Parinaz Fozouni et al. developed a CRISPR/Cas13a detection method that does not require amplification. The detection results can be read within 30 min using a smartphone microscope, which is more convenient and efficient.

Compared with conventional molecular diagnostic methods by PCR and quantitative polymerase chain reaction (qPCR), the CRISPR method shows advantages in sensitivity, case of operation and cost-effectiveness.

In view of the successful application of CRISPR-Cas13a system in the detection of a plurality of viruses mentioned above, it is hypothesized that the system will also be effective for detecting ALV-J. By designing specific crRNAs, a CRISPR-Cas13a system capable of recognizing and cleaving probe RNA is developed, providing a rapid, sensitive and economical detection method. It not only helps detect and control the spread of ALV-J in time, but also significantly reduces the detection cost, especially in remote areas with limited resources.

Therefore, it is urgent to develop a two-tube detection method utilizing the CRISPR system for the detection of ALV-J.

### SUMMARY

In view of this, an objective of the embodiments of the present disclosure is to provide a kit for rapid detection of ALV-J based on a CRISPR/Cas13a system. In the present disclosure, crRNA is designed with gp85 gene in ALV-J whole genome env sequence, so as to maximize the specificity of the gp85 gene in all ALV-J isolates or variants. A second objective of the embodiments of the present disclosure is to provide use of the kit in in vitro detection of ALV-J.

To achieve the above objectives, the present disclosure provides the following technical solutions.
1. Provided is a kit for rapid detection of ALV-J based on a CRISPR/Cas13a system. The kit includes a recombinase aided amplification (RAA) reagent and a CRISPR-Cas13a detection reagent for T7 transcriptases; where
   the RAA reagent includes a primer pair selected from the group consisting of a primer pair of SEQ ID NO: 1 and SEQ ID NO: 2, a primer pair of SEQ ID NO: 4 and SEQ ID NO: 5, and a primer pair of SEQ ID NO: 8 and SEQ ID NO: 9; and
   the CRISPR-Cas13a detection reagent for T7 transcriptases includes the T7 transcriptases, Cas13a, a fluorescent reporter gene, and crRNA primers; the fluorescent reporter gene has the sequence of SEQ ID NO: 23; and the crRNA primers are selected from the group consisting of a primer pair of SEQ ID NO: 13 and SEQ ID NO: 14, a primer pair of SEQ ID NO: 17 and SEQ ID NO: 18, and a primer pair of SEQ ID NO: 21 and SEQ ID NO: 22.

In some embodiments of the present disclosure, the RAA reagent further includes Reactive Powder, an RNA sample, an A buffer, and a B buffer.

In some embodiments of the present disclosure, the CRISPR-Cas13a detection reagent for T7 transcriptases further includes a buffer, an RNA inhibitor, ribonucleoside triphosphate (rNTP), 4-(2-hydroxyethyl)-1-piperazinecthanesulfonic acid (HEPES), MgCl₂, and RNA Target, and the RNA Target is an RAA product.

In some embodiments of the present disclosure, the kit further includes a lateral flow test strip including a conjugate pad zone, a test zone, and a control zone; the conjugate pad zone is immobilized with an RNA probe of 6-FAM-UUUUUUUUUUUUUU-C6 Biotin (SEQ ID NO: 24); the test zone includes a test (T) line immobilized with a capture molecule capable of specifically binding to C6 Biotin on the probe; after a target RNA-probe complex is captured, the test zone on the test strip forms a fluorescent line or a color line; the control zone includes a control (C) line immobilized with an anti-FAM antibody.

In some embodiments of the present disclosure, the capture molecule is selected from the group consisting of streptavidin and avidin.
3. Provided is use of the kit in in vitro detection of ALV-J.

Embodiments of the present disclosure have the following beneficial effects. The present disclosure provides a kit for rapid detection of ALV-J based on a CRISPR/Cas13a system. The method is based on the combination of a CRISPR/Cas13a system and RAA for the detection of ALV-J. An oligonucleotide probe is designed as a substrate for CRISPR/Cas13a trans-cleavage and produces a detectable signal. The method may substantially improve detection sensitivity by amplifying the detection signal twice by RAA and T7 transcription. Such a new detection method has a minimum limit of 5.4×10⁰ copies/μL. In addition, the detection method also exhibits excellent specificity, allowing for clear differentiation from other avian viruses. It does not require expensive experimental equipment and special laboratory environment, and it is both rapid and efficient. The method is of great significance for biological research and on-site detection of ALV-J.

### BRIEF DESCRIPTION OF THE DRAWINGS

In order to make the objectives, technical solutions and beneficial effects of the present disclosure clearer, the following drawings are provided.

FIG. 1 illustrates gel electrophoresis results using RAA (M: Marker; 1-5: amplification results for primers 1 to 5);

FIG. 2 illustrates determination results of purified LwaCas13 protein (1, 2, 3, 4, 5 and 6 represent different lanes with different proteins) by sodium dodecyl sulfate polyacrylamide gel electrophoresis (SDS-PAGE);

FIG. 3 illustrates the technological principle of two-step RAA-CRISPR/Cas13a detection; FIG. 4 illustrates continuous fluorescence monitored over 80 min;

FIGS. 5A-5B illustrate fluorescence intensities after the optimization of reaction time and temperature (FIG. 5A: time; FIG. 5B: temperature);

FIGS. 6A-6C illustrate fluorescence intensities after the optimization of crRNA concentration, Cas13a concentration and RAA products (FIG. 6A: crRNA concentration; FIG. 6B: Cas13a concentration; FIG. 6C: RAA product);

FIG. 7 illustrates assay results of lateral flow test strips;

FIG. 8 illustrates sensitivities of RAA-CRISPR/Cas13a to ALV-J; FIG. 9 illustrates detection sensitivities of lateral flow test strips;

FIG. 10 illustrates specificity results of a CRISPR/Cas13a detection system; and

FIGS. 11A-11B illustrate clinical samples detected by fluorescence and electrophoresis, respectively (A: amplication curves; FIG. 11A: electrophoresis results; FIG. 11B: fluorescence detection results).

### DETAILED DESCRIPTION OF THE EMBODIMENTS

The present disclosure will be further described in detail below in combination with the drawings and specific examples in order to enable those skilled in the art to better understand and implement the present disclosure. However, the illustrated examples should not be construed as limiting the present disclosure.

### Example 1. Design of RAA Primers and crRna

The sequence of envelope glycoprotein gp85 is the basis for the identification of ALV subgroups. In order to improve the specificity of RAA results, the gp85 sequences of ALV-J, ALV-A, ALV-B and ALV-K were downloaded from the National Center for Biotechnology Information (NCBI) (https://www.ncbi.nlm.nih.gov/), and aligned to select a conserved sequence of ALV-J using DNAstar (version 11.1.0) software. RAA primers containing T7 promoter were designed by Primer (https://blast.ncbi.nlm.nih.gov/). The primers were synthesized by Wuhan Tianyi Huayu Gene Technology Co., Ltd. (Wuhan, China). Specific sequences are shown in Table 1:

TABLE 1
Sequences of RAA primers

| Primer | Sequence | Length |
|---|---|---|
| RAA-Prime-1F | taatacgactcactatagggcaggtattttcttgatttgtggggacagg (SEQ ID NO. 1) | 49 |
| RAA-Prime-1R | gtggtaaagttaggagagagcatagtcaat (SEQ ID NO. 2) | 30 |
| RAA-Prime-2F | taatacgactcactatagggcaggtattttcttgatttgtggggacag (SEQ ID NO. 3) | 48 |
| RAA-Prime-2R | gagagagcatagtcaattgtcctagataa (SEQ ID NO. 4) | 30 |
| RAA-Prime-3F | taatacgactcactataggggtattttcttgatttgtggggacagg (SEQ ID NO. 5) | 46 |
| RAA-Prime-3R | gagagcatagtcaattgtcctagataacag (SEQ ID NO. 6) | 30 |
| RAA-Prime-4F | taatacgactcactatagggcaggtattttcttgatttgtggggac (SEQ ID NO. 7) | 46 |
| RAA-Prime-4R | agagagcatagtcaattgtcctagataaca (SEQ ID NO. 8) | 30 |
| RAA-Prime-5F | taatacgactcactataggggatattttagggtcccagatgatcaagaa (SEQ ID NO. 9) | 49 |
| RAA-Prime-5R | attaaaattcccatcaaaattgtgacagac (SEQ ID NO. 10) | 30 |
| RAA-Prime-6F | taatacgactcactatagggcaagaattggatattttagggtcccagatg (SEQ ID NO. 11) | 50 |
| RAA-Prime-6R | ttaaaattcccatcaaaattgtgacagac (SEQ ID NO. 12) | 30 |

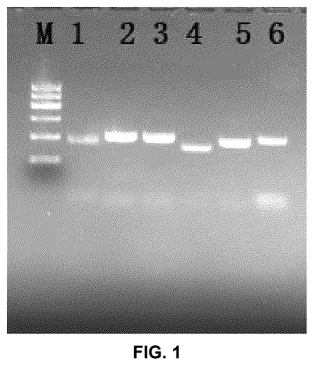

The designed primers were used for amplification, and the amplified products were detected by 2.5% agarose gel electrophoresis. The results are shown in FIG. 1. It was shown that the amplified product by RAAwas 116 bp in size, and the RAA products using primers 1, 3 and 5had higher brightness than those using other primers under gel imager, indicating that primers 1,3 and 5 were more suitable for detection.

According to the conserved sequence of the aligned ALV-J, the crRNA for detection was designed in the CHOPCHOP website. The crRNA was generated from the DNA template by in vitro transcription (IVT), and the T7 polymerase promoter sequence was attached to the 5′-end of the template. The DNA template of crRNA was synthesized by Wuhan Tianyi Huayu Gene Technology Co., Ltd. The DNA template of crRNA is shown in Table 2.

TABLE 2
The DNA template of crRNA

| Primers | Sequence | Size |
|---|---|---|
| crRNA-Prime-1F | Taatacgactcactataggggatttagactacccaaaaacgaaggggactaaaacgataacag | 82 |
| | ggccctcccaaggcattac (SEQ ID NO. 13) | |
| crRNA-Prime-1R | gtaatgccttgggagggccctgttatcttttagtccccttcgtttttggggtagtctaaatc | 62 |
| | (SEQ ID NO. 14) | |
| crRNA-Prime-2F | taatacgactcactataggggatttagactacccaaaaacgaaggggactaaaactaacagggc | 82 |
| | cctcccaaggcattacgc (SEQ ID NO. 15) | |
| crRNA-Prime-2R | gcgtaatgccttgggagggccctgttattttagtccccttcgtttttggggtagtctaaatc | 62 |
| | (SEQ ID NO. 16) | |
| crRNA-Prime-3F | taatacgactcactataggggatttagactacccaaaaacgaaggggactaaaaccagggccct | 82 |
| | cccaaggcattacgcggg (SEQ ID NO. 17) | |
| crRNA-Prime-3R | cccgcgtaatgccttgggagggccctgttttagtccccttcgtttttggggtagtctaaatc | 62 |
| | (SEQ ID NO. 18) | |
| crRNA-Prime-4F | taatacgactcactataggggatttagactacccaaaaacgaaggggactaaaacccctcccaa | 82 |
| | ggcattacgcgggatgcc (SEQ ID NO. 19) | |
| crRNA-Prime-4R | ggcatcccgcgtaatgccttgggagggttttagtccccttcgtttttggggtagtctaaatc | 62 |
| | (SEQ ID NO. 20) | |
| crRNA-Prime-5F | taatacgactcactataggggatttagactacccaaaaacgaaggggactaaaacaacagggc | 82 |
| | cctcccaaggcattacgcg (SEQ ID NO. 21) | |
| crRNA-Prime-5R | cgcgtaatgccttgggagggccctgttttttagtccccttcgtttttggggtagtctaaatc | 62 |
| | (SEQ ID NO. 22) | |

### Example 2. Preparation of crRNA and RNA Reporter

In order to generate double-stranded DNA, crRNA oligonucleotides containing the T7 promoter in Table 2 were annealed to dsDNA using Annealing Buffer for DNA Oligos (TAKARA®, China) and HiScribe T7 Quick High Yield RNA Synthesis Kit (New England Biolabs (NEB®), Ipswich, MA, USA). The synthesized dsDNA was purified using a DNA Purification Kit (TAKARA®, China), and the transcribed crRNA was purified using an RNA Purification Kit (Sangon®, Shanghai, China). RNA concentration was measured using NanoDrop® One, and the purified crRNA was held at −80° C. until use.

Fluorescent Reporter RNA labeled with FAM and BHQ1 at both ends was synthesized by Guangzhou Editgene Co., Ltd., China. Reporter RNA labeled with FAM and Biotin at both ends was synthesized by Wuhan Tianyi Huayu Gene Technology Co., Ltd. Specific sequences are shown in Table 3.

TABLE 3
Primes for ssRNA Reporter

| SSRNA Reporter | Sequence |
|---|---|
| FAM Reporter | FAM-UUUUUU-BHQ1 |
| Bioin Reporter | 6-FAM-UUUUUUUUUUUUUU-C6 Biotin |
| | (SEQ ID NO: 24) |

### Example 3. Purification and Large-Scale Expression of LwaCas13 Protein

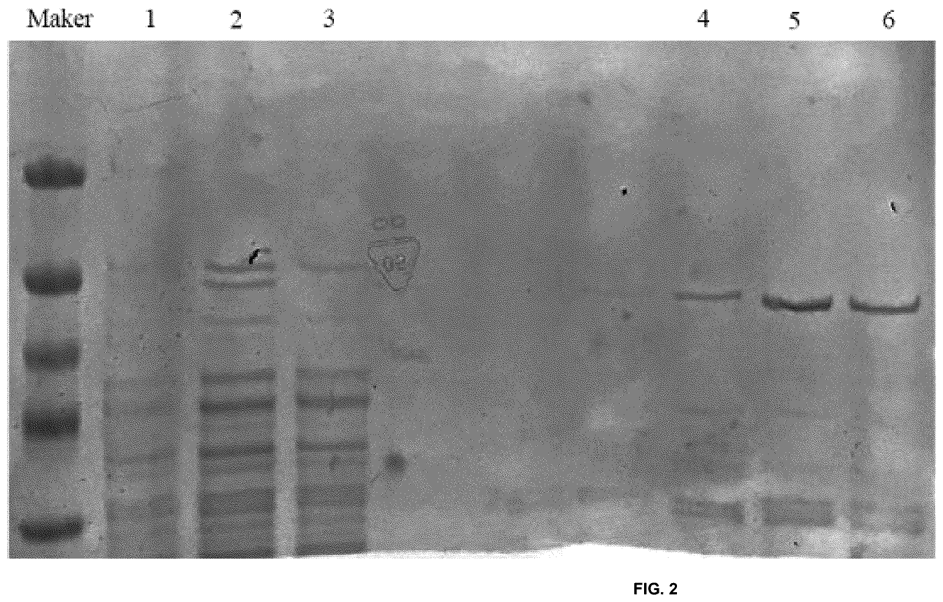

The His-tagged and SUMO-tagged LwaCas13 protein required a series of purification and detagging. The plasmid pC013-Twinstrep-SUMO-hμlwCas13a (Wuhan Miaoling Biotechnology Co., Ltd., China) containing the inserted LwaCas13a gene was transformed into Escherichia coliBL21 (DE3). Low temperature induced expression was conducted at 16° C. for 14 h by adding 1 mL/L 0.5M IPTG, and the cell suspension obtained after induced expression was disrupted under an ultrasonic cell disrupter, and centrifuged at 8,000 rpm for 30 min at 4° C. to collect a supernatant containing LwaCas13. The supernatant was filtered using a 22 μm filter, and then the LwaCas13a protein was purified using a NI-NTA prepacked gravity column. Impure proteins were gradiently cluted using 20, 50, and 80 mM imidazole, and Cas13 proteins bound to the NI column was eluted using 500 mM imidazole. The solution containing LwaCas13 protein was collected. The SUMO tag was detagged overnight at 4° C. using 1.5% SUMO protease. The collected eluent was concentrated with an ultrafiltration tube (Millipore®, Germany), and finally the purified LwaCas13 protein was determined by SDS-PAGE and Coomassie brilliant blue staining. The results are shown in FIG. 2. The results showed that highly pure and active Cas13a protein was successfully obtained. The protein was quantified by BCA kit (Beyotime) and stored at −80° C. for later use.

### Example 4. Preparation of Virus

Liver, plasma and cloacal samples were collected from poultry individuals manifested with symptoms of avian leukosis in a chicken farm in Chongqing. The results of virus isolation and identification provided by the Avian Leukosis Laboratory of the College of Animal Science and Technology of Southwest University showed that the positive samples contained ALV-J. Viral RNAs were successfully extracted according to the instructions of the manufacturer of the MagicPure®Simple Viral DNA/RNA Kit. Subsequently, these RNAs were reversely transcribed into cDNAs for subsequent experiments. At the same time, the DNA samples were properly stored at −20° C. until further use.

### Example 5. Construction of Standard Plasmid

In this study, in order to explore the sensitivity of the system to detect the virus, the above reference gp85 sequence of ALV-J isolate was obtained. The specific sequence is set forth in SEQ ID NO: 23:

ggagttcatctgttgcaacaaccagggaacgtatgggtcacctgggcaa
ataagacgggccaaacagatttttgccttagtctacagtcggctacctc
tcccttccgcacctgtttggtgggtttgccgagttaccaattggaggag
tttagaggatatacggtcaactacactggatgtaaaaatgatacagatg
cggccacacagacggcgtgtctgattcaatcattaaatcgtaccctccc
ttgggacccccaagaattggatattttagggtcccagatgatcaagaac
ggaacaacacgtacgtgtgttacctttggttcggtgtgctataaagaga
acaatcgcagtagagtctgtcacaattttgatgggaattttaatgggac
tggtggggcagaagcagaattgcgtgacttcatagcaaaatggaaaagt
gatgaccttcttataagaccctatgtcaaccaatcatggacgatggtaa
gtccaataaacgtagagagtttttcaataagtcgtagatattgtggatt
caccagtaacgagactcgttactatagaggggacctttctaattggtgt
ggttcaaaaaggggaaaatggtcagcggggtacagcaacgggacaaaat
gttccagcaacacgacgggttgcggtggtaattgcacgacggaatggaa
ttattatgcatatgggtttaccttcgggaaacagccagaggtgttgtgg
aacaatgggactgctaaggcactccccccaggtattttcttgatttgtg
gggacagggcttggcaaggcatcccgcgtaatgccttgggagggccctg
ttatctaggacaattgactatgctctctccTaactttaccacctggata
acatatgggccgaacattacgggtcaccgccgtagcaggcgc

The gp85 sequence was synthesized by Wuhan Genecreate Biological Engineering Co., Ltd., and then cloned into a PUC-57 vector to obtain gp85-PUC-57.

### Example 6: Two-Step RAA-CRISPR/Cas13a Detection

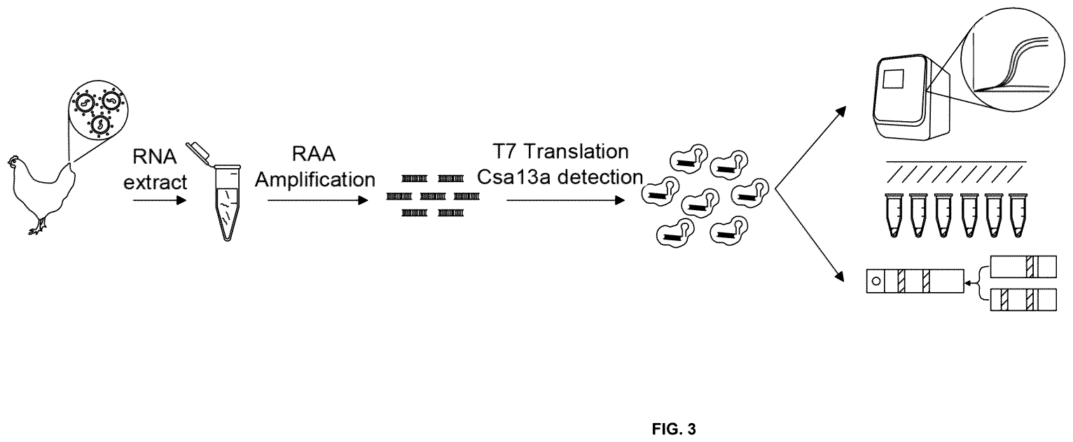

This study adopted a two-step RAA-CRISPR/Cas13a detection technology, the principle of which is shown in FIG. 3. The technology included an RAA process and a CRISPR-Cas13a detection system containing the T7 transcriptases. According to the instructions of the kit manufacturer (the reagent was Basic Nucleic Acid Amplification Reagent (RAA method) purchased from Hangzhou ZC Bio-Sci & Tech Co., Ltd.), the premix was first prepared on ice. The premix contained 2 μL of Forward Primer (RAA-Prime-3F) (10 μM), 2 μL of Reverse Primer (RAA-Prime-3R) (10 μM), 1 μL of ReactivePowder, 5 μL of RNA sample, 25 μL of A buffer, and 2.5 μL of B buffer. Then the premix was quickly vortexed and mixed. Finally, the reaction was conducted in a metal bath at 42° C. for 30 min.

T7 transcription is essential for CRISPR-Cas13a detection. Briefly, 0.2 μL of 50 nM LwaCas13a, 4 μL of Buffer, 1 μL of 500 nM crRNA, 1.25 μL of 2 μM ssRNA Reporter, 1 μL of 40 U RNA Inhibitor, 0.6 μL of 1 μM RNA Targrt, 0.8 μL of rNTP mixture/25 mM, 0.4 μL of HEPES/1M, 0.18 μL of MgCl₂/1M, 0.5 μL of T₇ transcriptases/5 U, and 10.07 μL of H₂O were mixed, with a total volume of 20 μL, where crRNA was finally added to activate Cas13a protein activity. In order to monitor the reaction, the mixture was incubated at 37° C. and the fluorescence intensity was recorded every 30 s for 80 min to collect data. The results are shown in FIG. 4. The results showed that the fluorescence intensity remained unchanged when the detection system did not contain any of the following components: Cas13a protein, crRNA or RAA product. When

Cas13a protein, crRNA and RAA product were present at the same time, the fluorescence intensity increased significantly. The results showed that it was only in the complete system that the target RNA of ALV-J could activate the Cas13a/crRNA trans-cleavage system.

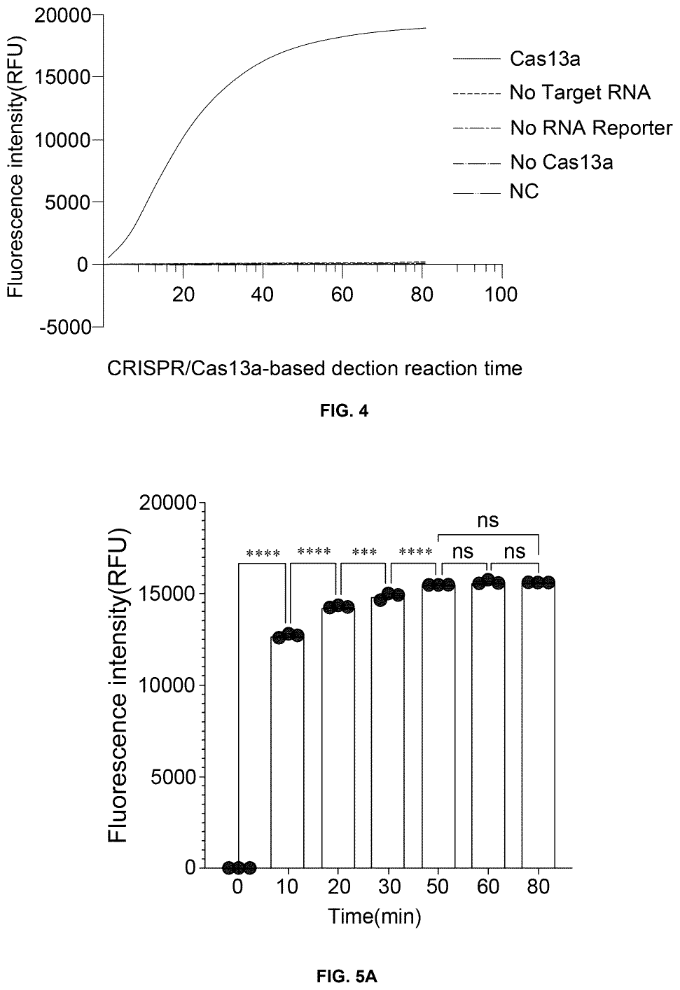

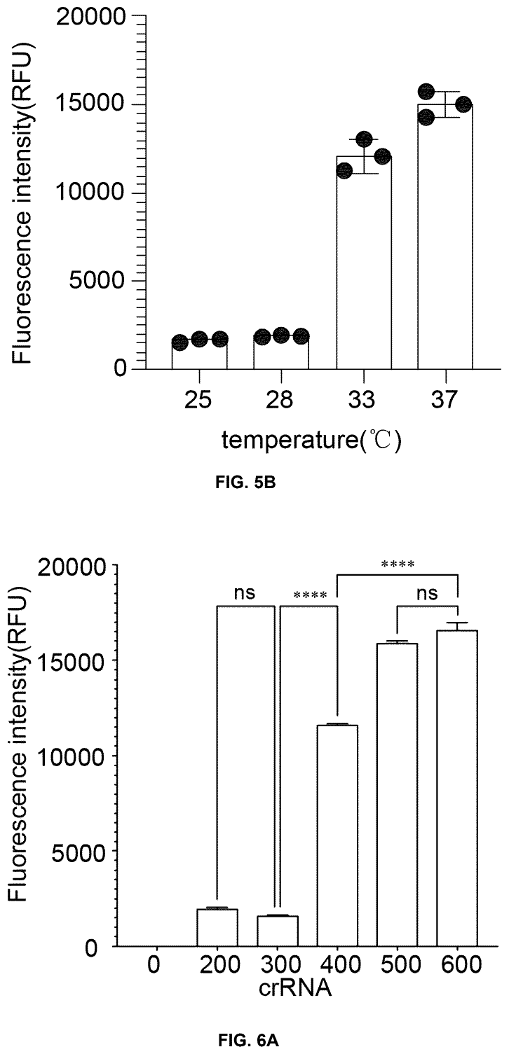

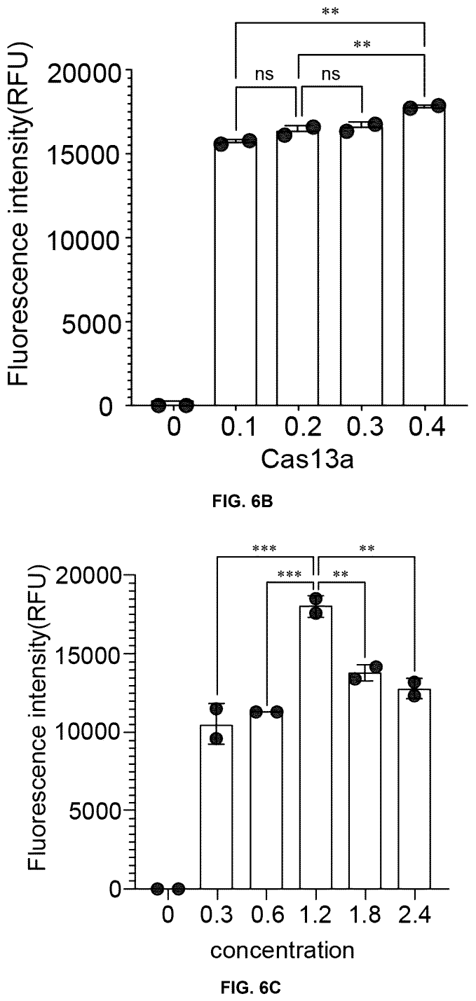

Since the Cas13a protein was involved in the detection reaction as an enzyme, the performance of the detection system was closely related to other parameters such as reaction time, temperature, enzyme concentration, target RNA, etc. Therefore, some reaction conditions were optimized in order to achieve the best detection effect. The reaction time of CRISPR-Cas13a system was set at 10, 20, 30, 50, 60 and 80 min, respectively, and the temperature was set at 25, 28, 33 and 37° C., respectively. The results are shown in FIGS. 5A-5B. The results showed that the fluorescence intensity reached the maximum at 37° C. and 30 min. In addition, concentrations of crRNA, Cas13a and RAA product were also optimized according to fluorescence intensity. The results are shown in FIGS. 6A-6C. The results showed that strong fluorescence were generated when the crRNA concentration was higher than 400 nM and the Cas13a concentration was higher than 0.1 nM, while the fluorescence intensity of the RAA product was the highest at a concentration of 1.2 μL.

The lateral flow test strip was constructed to verify the CRISPR-Cas13a detection ALV-J system. The specific method was as follows:
(1) Preparation of sample mixture:
   The test sample was mixed with other reagents required for the reaction (such as RAA reagent and CRISPR-Cas13a system), and finally diluted with diethyl pyrocarbonate (DEPC) water to a total volume of 50 μL.

Preparation of lateral flow test strip: The RNA probe 6-FAM-UUUUUUUUUUUUUU-C6 Biotin (SEQ ID NO: 24) was immobilized on the conjugate pad zone of the test strip. The polyuridine moiety (UUUUUUUUUUUUUU, SEQ ID NO: 24) of the probe was complementary to the target RNA in the sample to form an RNA-probe complex.

The test zone included a T line, which was immobilized with capture molecules such as streptavidin or avidin. These capture molecules could specifically bind to C6 Biotin (biotin-labeled) on the probe. When the target RNA-probe complex was captured, the test zone of the test strip formed a fluorescent line or a color line. The intensity of this signal is related to the concentration of the target RNA. The probe that did not bind to the target RNA continued to flow forward and entered the control zone. The control zone is usually immobilized with another binding molecule, such as anti-6-FAM antibody or other capture molecules, which bind to the 6-FAM moiety on the probe to form a C line.

During the detection, the test strip was let stand and its lower section was guaranteed to be always immersed in the mixture for 10 min. After 10 min, the results on the test strip were observed, and the results beyond 10 min were ineffective.

Result determination: A positive result could be determined only when the control (C) line and the test (T) line on the test strip were developed at the same time.

If only the C line is developed and the T line is not, or if there is no color, it is determined as a negative result.

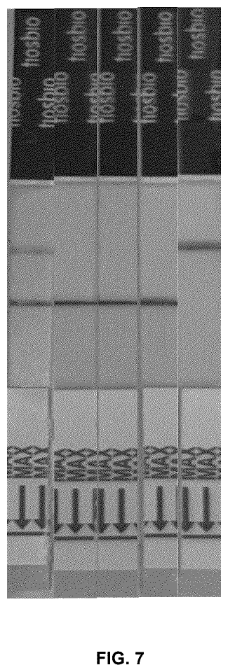

The detection results are shown in FIG. 7. The results showed that the first test strip was tested positive, and the rest were tested negative.

### Example 7. Sensitivity of CRISPR/Cas13a System to Detect ALV-J

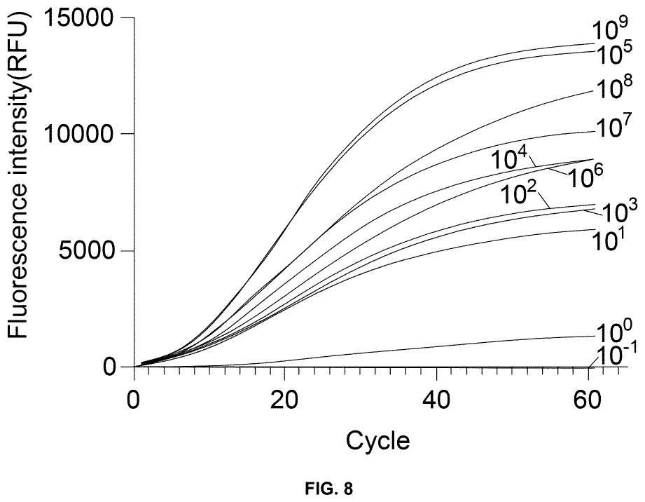

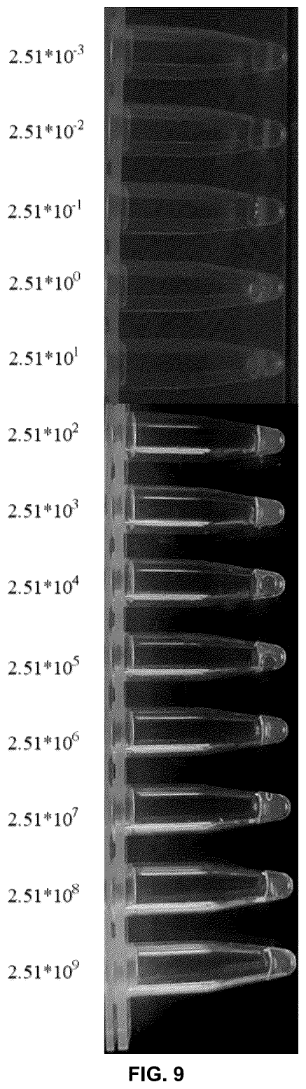

The minimum limit of the system in ALV-J detection was explored by using the optimized experimental conditions. The plasmid DNA with an initial concentration of 2.51×10⁹ copies/μL was diluted 10 times with RNase-free water to produce a concentration gradient. The sensitivity of RAA-CRISPR/Cas13a to ALV-J was evaluated by this gradient. The results are shown in FIG. 8. The results showed that more ssRNAs could be obtained with the increase of ALV-J concentration, and thus more collateral cleavage of Cas13a was activated, resulting in a significant increase in fluorescence.

The viral RNAs with concentrations of 10⁹, 10⁸, 10⁷, 10⁶, 10⁵, 10⁴, 10³, 10², 10¹, and 10⁰ were detected by lateral flow test strips. The results are shown in FIG. 9. The results showed that the CRISPR/Cas13a detection system could detect as low as 5.4×10⁰ copies of viral RNAs, demonstrating the superior performance of the CRISPR/Cas13a system in low-concentration virus detection.

### Example 8. Specificity of CRISPR-Cas13a to Detect ALV-J

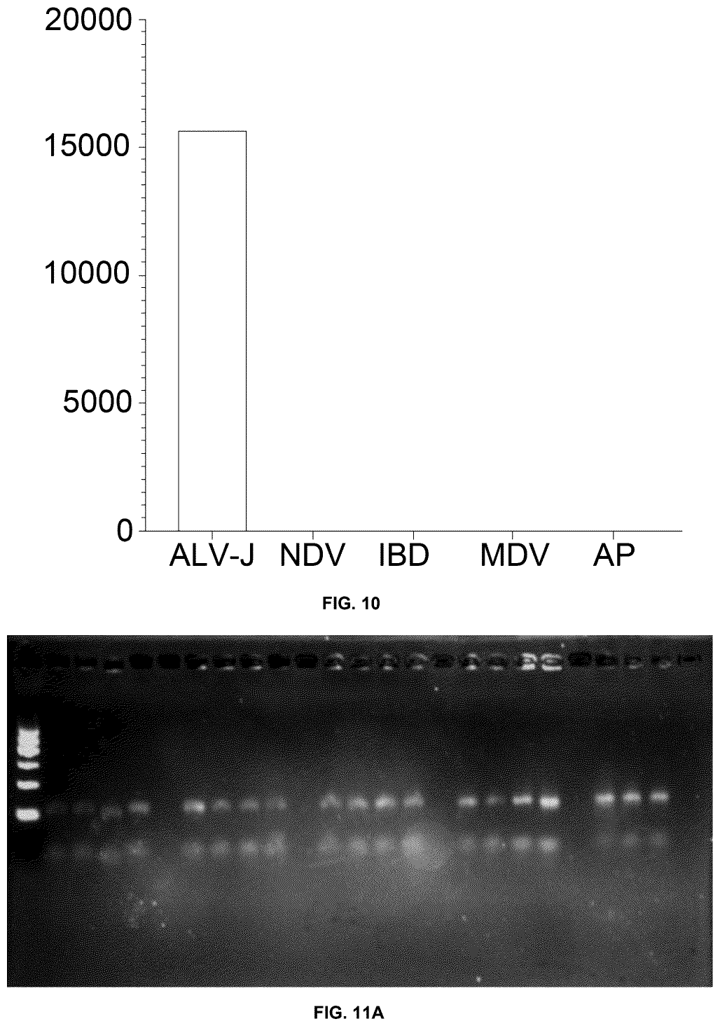

An excellent avian leukosis virus detection method can distinguish different avian viruses (NDV, IBD, MDV, and AP). The specificity of the CRISPR/Cas13a detection system was evaluated by five different avian viruses. As shown in FIG. 10, at the same initial concentration (100 nM), the change of fluorescence intensity in the same system is not significantly different from that of the blank control, and only ALV-JRNA produces significant signal enhancement. These results indicate that the detection method has excellent specificity.

### Example 9. Detection of Clinical Samples of ALV-J Using CRISPR-Cas13a

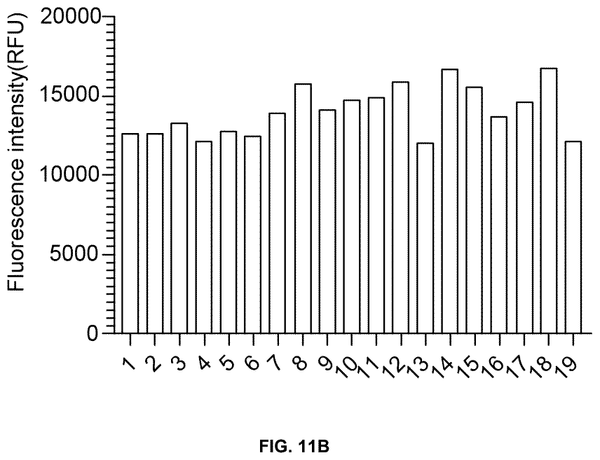

In order to further verify the applicability and accuracy of the CRISPR/Cas13a system in detecting target RNA in complex RNA extracts, the extraction of viral RNAs from 300 blood samples collected from different chicken farms was analyzed by Cas13a fluorescence detection. Viral RNAs were detected by fluorescence detection method and electrophoresis, respectively. The results are shown in FIGS. 11A-11C. As shown in the figure, there is a significant difference in the results between the experimental group and the control group. The experimental results show that the detection method can accurately identify the target RNA in complex RNA samples. Therefore, the detection method proposed in this experiment is the most powerful tool for detecting ALV-J in complex substrates.

The foregoing examples are only preferred embodiments illustrated for fully explaining the present disclosure, and the claimed scope of the present disclosure is not limited thereto. Equivalent substitutions or transformations made by those skilled in the art on the basis of the present disclosure are within the claimed scope of the present disclosure. The claimed scope of the present disclosure shall be subject to the claims.

What is claimed is:

1. A kit for rapid detection of avian leukosis virus subgroup J (ALV-J) based on a CRISPR/Cas13a system, wherein the kit comprises a recombinase aided amplification (RAA) reagent and a CRISPR-Cas13a detection reagent for T7 transcriptases;
the RAA reagent comprises a primer pair selected from the group consisting of a primer pair of SEQ ID NO: 1 and SEQ ID NO: 2, a primer pair of SEQ ID NO: 4 and SEQ ID NO: 5, and a primer pair of SEQ ID NO: 8 and SEQ ID NO: 9; and
the CRISPR-Cas13a detection reagent for T7 transcriptases comprises the T7 transcriptases, Cas13a, a fluorescent reporter gene, and crRNA primers; the fluorescent reporter gene has a sequence of SEQ ID NO: 23; and the crRNA primers are selected from the group consisting of a primer pair of SEQ ID NO: 13 and SEQ ID NO: 14, a primer pair of SEQ ID NO: 17 and SEQ ID NO: 18, and a primer pair of SEQ ID NO: 21 and SEQ ID NO: 22.

2. The kit for rapid detection of ALV-J based on a CRISPR/Cas13a system according to claim 1, wherein the RAA reagent further comprises Reactive Powder, an RNA sample, an A buffer, and a B buffer.

3. The kit for rapid detection of ALV-J based on a CRISPR/Cas13a system according to claim 1, wherein the CRISPR-Cas13a detection reagent for T7 transcriptases further comprises a Buffer, an RNA inhibitor, ribonucleoside triphosphate (rNTP), 4-(2-hydroxyethyl)-1-piperazineethanesulfonic acid (HEPES), MgCl₂, and RNA Target, and the RNA Target is an RAA product.

4. The kit for rapid detection of ALV-J based on a CRISPR/Cas13a system according to claim 1, wherein the kit further comprises a lateral flow test strip comprising a conjugate pad zone, a test zone, and a control zone; the conjugate pad zone is immobilized with an RNA probe of 6-FAM-UUUUUUUUUUUUUU-C6 Biotin (SEQ ID NO: 24); the test zone comprises a test (T) line immobilized with a capture molecule capable of specifically binding to C6 Biotin on the probe; after a target RNA-probe complex is captured, the test zone on the test strip forms a fluorescent line or a color line; the control zone comprises a control (C) line immobilized with an anti-FAM antibody.

5. The kit for rapid detection of ALV-J based on a CRISPR/Cas13a system according to claim 4, wherein the capture molecule is one selected from the group consisting of streptavidin and avidin.

6. A method for in in vitro detection of ALV-J, comprising contacting a specimen with the kit according to claim 1.

7. The method according to claim 6, wherein the RAA reagent further comprises Reactive Powder, an RNA sample, an A buffer, and a B buffer.

8. The method according to claim 6, wherein the CRISPR-Cas13a detection reagent for T7 transcriptases further comprises a Buffer, an RNA inhibitor, ribonucleoside triphosphate (rNTP), 4-(2-hydroxyethyl)-1-piperazineethanesulfonic acid (HEPES), MgCl2, and RNA Target, and the RNA Target is an RAA product.

9. The method according to claim 6, wherein the kit further comprises a lateral flow test strip comprising a conjugate pad zone, a test zone, and a control zone; the conjugate pad zone is immobilized with an RNA probe of 6-FAM-UUUUUUUUUUUUUU-C6 Biotin (SEQ ID NO: 24); the test zone comprises a test (T) line immobilized with a capture molecule capable of specifically binding to C6 Biotin on the probe; after a target RNA-probe complex is captured, the test zone on the test strip forms a fluorescent line or a color line; the control zone comprises a control (C) line immobilized with an anti-FAM antibody

10. The method according to claim 9, wherein the capture molecule is one selected from the group consisting of streptavidin and avidin.


SEQUENCE LISTING (24 sequences)

SEQ ID NO: 1 - DNA, 49 bp, synthetic construct
  source 1..49: mol_type = other DNA; note = Primer RAA-Prime-1F
taatacgactcactatagggcaggtattttcttgatttgtggggacagg

SEQ ID NO: 2 - DNA, 30 bp, synthetic construct
  source 1..30: mol_type = other DNA; note = Primer RAA-Prime-1R
gtggtaaagttaggagagagcatagtcaat

SEQ ID NO: 3 - DNA, 48 bp, synthetic construct
  source 1..48: mol_type = other DNA; note = Primer RAA-Prime-2F
taatacgactcactatagggcaggtattttcttgatttgtggggacag

SEQ ID NO: 4 - DNA, 29 bp, synthetic construct
  source 1..29: mol_type = other DNA; note = Primer RAA-Prime-2R
gagagagcatagtcaattgtcctagataa

SEQ ID NO: 5 - DNA, 46 bp, synthetic construct
  source 1..46: mol_type = other DNA; note = Primer RAA-Prime-3F
taatacgactcactataggggtattttcttgatttgtggggacagg

SEQ ID NO: 6 - DNA, 30 bp, synthetic construct
  source 1..30: mol_type = other DNA; note = Primer RAA-Prime-3R
gagagcatagtcaattgtcctagataacag

SEQ ID NO: 7 - DNA, 46 bp, synthetic construct
  source 1..46: mol_type = other DNA; note = Primer RAA-Prime-4F
taatacgactcactatagggcaggtattttcttgatttgtggggac

SEQ ID NO: 8 - DNA, 30 bp, synthetic construct
  source 1..30: mol_type = other DNA; note = Primer RAA-Prime-4R
agagagcatagtcaattgtcctagataaca

SEQ ID NO: 9 - DNA, 49 bp, synthetic construct
  source 1..49: mol_type = other DNA; note = Primer RAA-Prime-5F
taatacgactcactataggggatattttagggtcccagatgatcaagaa

SEQ ID NO: 10 - DNA, 30 bp, synthetic construct
  source 1..30: mol_type = other DNA; note = Primer RAA-Prime-5R
attaaaattcccatcaaaattgtgacagac

SEQ ID NO: 11 - DNA, 50 bp, synthetic construct
  source 1..50: mol_type = other DNA; note = Primer RAA-Prime-6F
taatacgactcactatagggcaagaattggatattttagggtcccagatg

SEQ ID NO: 12 - DNA, 29 bp, synthetic construct
  source 1..29: mol_type = other DNA; note = Primer RAA-Prime-6R
ttaaaattcccatcaaaattgtgacagac

SEQ ID NO: 13 - DNA, 82 bp, synthetic construct
  source 1..82: mol_type = other DNA; note = Primer crRNA-Prime-1F
taatacgactcactataggggatttagactacccaaaaacgaaggggactaaaacgataacagggccctcccaaggcattac

SEQ ID NO: 14 - DNA, 62 bp, synthetic construct
  source 1..62: mol_type = other DNA; note = Primer crRNA-Prime-1R
gtaatgccttgggagggccctgttatcttttagtccccttcgtttttggggtagtctaaatc

SEQ ID NO: 15 - DNA, 82 bp, synthetic construct
  source 1..82: mol_type = other DNA; note = Primer crRNA-Prime-2F
taatacgactcactataggggatttagactacccaaaaacgaaggggactaaaactaacagggccctcccaaggcattacgc

SEQ ID NO: 16 - DNA, 62 bp, synthetic construct
  source 1..62: mol_type = other DNA; note = Primer crRNA-Prime-2R
gcgtaatgccttgggagggccctgttattttagtccccttcgtttttggggtagtctaaatc

SEQ ID NO: 17 - DNA, 82 bp, synthetic construct
  source 1..82: mol_type = other DNA; note = Primer crRNA-Prime-3F
taatacgactcactataggggatttagactacccaaaaacgaaggggactaaaaccagggccctcccaaggcattacgcggg

SEQ ID NO: 18 - DNA, 62 bp, synthetic construct
  source 1..62: mol_type = other DNA; note = Primer crRNA-Prime-3R
cccgcgtaatgccttgggagggccctgttttagtccccttcgtttttggggtagtctaaatc

SEQ ID NO: 19 - DNA, 82 bp, synthetic construct
  source 1..82: mol_type = other DNA; note = Primer crRNA-Prime-4F
taatacgactcactataggggatttagactacccaaaaacgaaggggactaaaacccctcccaaggcattacgcgggatgcc

SEQ ID NO: 20 - DNA, 62 bp, synthetic construct
  source 1..62: mol_type = other DNA; note = Primer crRNA-Prime-4R
ggcatcccgcgtaatgccttgggagggttttagtccccttcgtttttggggtagtctaaatc

SEQ ID NO: 21 - DNA, 82 bp, synthetic construct
  source 1..82: mol_type = other DNA; note = Primer crRNA-Prime-5F
taatacgactcactataggggatttagactacccaaaaacgaaggggactaaaacaacagggccctcccaaggcattacgcg

SEQ ID NO: 22 - DNA, 62 bp, synthetic construct
  source 1..62: mol_type = other DNA; note = Primer crRNA-Prime-5R
cgcgtaatgccttgggagggccctgttttttagtccccttcgtttttggggtagtctaaatc

SEQ ID NO: 23 - DNA, 924 bp, synthetic construct
  source 1..924: mol_type = other DNA; note = gp85 sequence of ALV-J
ggagttcatctgttgcaacaaccagggaacgtatgggtcacctgggcaaataagacgggccaaacagatttttgccttagtctacagtcggctacctctcccttccgcacctgtttggtgggtttgccgagttaccaattggaggagtttagaggatatacggtcaactacactggatgtaaaaatgatacagatgcggccacacagacggcgtgtctgattcaatcattaaatcgtaccctcccttgggacccccaagaattggatattttagggtcccagatgatcaagaacggaacaacacgtacgtgtgttacctttggttcggtgtgctataaagagaacaatcgcagtagagtctgtcacaattttgatgggaattttaatgggactggtggggcagaagcagaattgcgtgacttcatagcaaaatggaaaagtgatgaccttcttataagaccctatgtcaaccaatcatggacgatggtaagtccaataaacgtagagagtttttcaataagtcgtagatattgtggattcaccagtaacgagactcgttactatagaggggacctttctaattggtgtggttcaaaaaggggaaaatggtcagcggggtacagcaacgggacaaaatgttccagcaacacgacgggttgcggtggtaattgcacgacggaatggaattattatgcatatgggtttaccttcgggaaacagccagaggtgttgtggaacaatgggactgctaaggcactccccccaggtattttcttgatttgtggggacagggcttggcaaggcatcccgcgtaatgccttgggagggccctgttatctaggacaattgactatgctctctcctaactttaccacctggataacatatgggccgaacattacgggtcaccgccgtagcaggcgc

SEQ ID NO: 24 - RNA, 14 nt, synthetic construct
  source 1..14: mol_type = other RNA; note = RNA probe immobilized on the conjunction pad zone
tttttttttttttt